(19) United States
(12) Patent Application Publication
(10) Pub. No.: US 2011/0269248 A1
(43) Pub. Date: Nov. 3, 2011

(54) BIO-MARKERS FOR DIAGNOSING DIABETIC RETINOPATHY

(75) Inventors: Chan Wha KIM, Seoul (KR); Hyunsyuk Yoo, Seoul (KR); Jae-Chan Kim, Seoul (KR); Pan Kyeom Kim, Seoul (KR); Hye Won Park, Seoul (KR); Mi Ryung Kim, Goyang-si (KR)

(73) Assignee: Korea University Research and Business Foundation, Seoul (KR)

(21) Appl. No.: 13/182,915

(22) Filed: Jul. 14, 2011

Related U.S. Application Data
(62) Division of application No. 12/354,907, filed on Jan. 16, 2009.

(30) Foreign Application Priority Data
Jan. 17, 2008 (KR) 10-2008-0005162

(51) Int. Cl. G01N 33/53

(52) U.S. Cl. 436/501

(57) ABSTRACT

Disclosed herein are bio-markers for diagnosing diabetic retinopathy, a use of proteins, whose expression level down-regulated or up-regulated in the tears of a non-proliferative diabetic retinopathy (NPDR) patient, as bio-markers for diagnosing diabetic retinopathy, and a composition and kit for diagnosing diabetic retinopathy comprising antibodies against said proteins.

### CROSS-REFERENCE TO RELATED APPLICATIONS

This application is a divisional of U.S. patent application Ser. No. 12/354,907 filed Jan. 16, 2009, which claims priority based on Korean Patent Application No. 10-2008-0005162, filed Jan. 17, 2008, the contents of all of which are incorporated herein by reference in their entirety.

### FIELD OF THE INVENTION

The present invention relates to bio-markers for diagnosing diabetic retinopathy; and, more specifically, it pertains to a use of proteins, whose expression level down-regulated or up-regulated in the tears of a non-proliferative diabetic retinopathy (NPDR) patient, as bio-markers for diagnosing diabetic retinopathy, and a composition and kit for diagnosing diabetic retinopathy comprising antibodies against said proteins.

### BACKGROUND OF THE INVENTION

Diabetes mellitus (DM), a representative metabolic disorder caused by long-term maintenance of abnormally high blood glucose (sugar) level, is a systemic, non-contagious, chronic disease.

Diabetes mellitus develops due to an absolutely or relatively diminished production of insulin induced by autoimmune destruction of insulin-producing beta cells of the pancreas, or decreased effects of insulin in a target organ (insulin resistance), which causes consistent increase of blood glucose level and impairs body metabolism including glucose metabolism.

Diabetic retinopathy is one of the major microvascular complications of diabetes mellitus, which is caused by inharmonious supply of blood to the retina due to the circulatory disturbance resulting from the gradual deformation and occlusion of the retinal microvessels on account of the consistently high blood glucose level and metabolic abnormality caused thereby. Because the frequency of occurrence of diabetic retinopathy increases as the period suffering from diabetes mellitus is long and there are no warning signs for some time, it is necessary to periodically examine the change of the retina in a diabetic patient.

Recently, there have been increasing needs for developing diagnostic markers for the early diagnosis of diabetic retinopathy and an effective method for diagnosing diabetic retinopathy. Accordingly, many proteins have been presented as markers for diagnosing diabetic retinopathy, examples of such proteins including vascular adhesion molecules such as endothelin-1 (ET-1), protein kinase C (PKC) and E-selectin; thrombin-cleaved osteopontin; phosphoinositides; thrombin-stimulated platelet aggregation; vitreous VEGF; circulating soluble ICAM-1 (sICAM-1); advanced glycation end products (AGEs); nitric oxide; cytokines such as IL-1, IL-6, IL-8 and tumor necrosis factor-α; HLA alleles such as HLA-DR1, HLA-A9 and HLA-B40; glycosylated haemoglobin (HbAlc); microalbuminurea; aldose reductase (AR); Fas; PEDF; and the like proteins. However, none of the above proteins showed specificity for diabetic retinopathy only.

Hitherto, a serum has been typically used as a source of biomarkers for the early diagnosis of diabetic retinopathy. However, there have been difficulties in finding out such biomarkers from a serum because of the glycosylation of serum proteins and presence of other abundant proteins such as albumin and immunoglobulin.

Generally, tears consist of mucins, lipids, salts, glycoproteins and other various proteins. Representative tear proteins include lysozyme, lactoferrins, secretory immunoglobulin A (sIgA), lipocalins, albumins and lipophilins. Any quantitative or qualitative changes of these proteins may be interpreted as changes in the pathological conditions of our bodies. Although there have been many attempts to analyze tear proteins in relation with a disease, most of them are limited to discover the relationship between a single protein with a specific disease.

The present inventors have endeavored to analyze the tear proteins more accurately and efficiently, and to develop effective biomarkers for early diagnosis of diabetic retinopathy. Finally, the present inventors have succeeded in accurately and efficiently analyzing tear proteins by employing two-dimensional gel electrophoresis (2-DE), thereby finding out many biomarker proteins for the diagnosis of diabetic retinopathy. Further, the present inventors have developed a composition for treating or preventing diabetic retinopathy, which comprises the biomarker proteins, and a composition and a kit for diagnosing diabetic retinopathy, which comprises an antibody specific for any one of the biomarker proteins.

### SUMMARY OF THE INVENTION

Accordingly, it is an object of the present invention to provide proteins or immunogenic fragments thereof useful for diagnosing diabetic retinopathy.

It is another object of the present invention to provide a composition for diagnosing diabetic retinopathy, comprising antibodies specific for the proteins or the immunogenic fragments thereof.

It is a further object of the present invention to provide a kit for diagnosing diabetic retinopathy, comprising antibodies specific for the protein or the immunogenic fragments thereof.

It is a still further object of the present invention to provide a composition for treating or preventing diabetic retinopathy, comprising the proteins or the immunogenic fragments thereof as an active ingredient.

In accordance with one aspect of the present invention, there is provided proteins, or immunogenic fragments thereof for diagnosing diabetic retinopathy.

In accordance with another aspect of the present invention, there is provided a composition for diagnosing diabetic retinopathy, comprising antibodies specific for the proteins or the immunogenic fragments thereof.

In accordance with a further aspect of the present invention, there is provided a kit for diagnosing diabetic retinopathy, comprising antibodies specific for the proteins or the immunogenic fragments thereof.

In accordance with a still further aspect of the present invention, there is provided a composition for treating or preventing diabetic retinopathy, comprising the proteins or the immunogenic fragments thereof as an active ingredient.

### BRIEF DESCRIPTION OF THE DRAWINGS

The above and other objects and features of the present invention will become apparent from the following description of the invention, when taken in conjunction with the accompanying drawings, which respectively show:

FIG. 1A is a 2-DE gel image of the proteins expressed in the tears of healthy person;

FIGS. 1B and 1C is 2-DE gel images of the proteins down-regulated or up-regulated in the tears of a non-retinopathic diabetic patient and an NPDR patient, respectively;

FIG. 2A is magnified images of the proteins shown in FIGS. 1A to 1C;

FIG. 2B is relative intensities of the proteins shown in FIGS. 1A to 1C;

FIG. 3A is a western blot analysis result of LCN-1, Hsp27 and B2M identified in the tears of a patient with diabetic retinopathy; and

FIGS. 3B to 3D display relative intensities of LCN-1, Hsp27 and B2M, respectively, in healthy persons, non-retinopathic diabetic patients and NPDR patients.

In the above figures, Healthy means a healthy person; No DMR, a non-retinopathic diabetic patient; and NPDR, a NPDR patient;

### DETAILED DESCRIPTION OF THE INVENTION

In accordance with one aspect of the present invention, there is provided a use of a protein selected from the group consisting of the following proteins or an immunogenic fragment thereof as a biomarker for diagnosing diabetic retinopathy:

DJ-1 protein having molecular weight of 24 to 26 kDa and isoelectric point (PI) of 5.83 to 6.83, which comprises peptide sequences of SEQ ID NOs: 1 and 2 obtained by trypsin digestion thereof;

beta 2-microglobulin (B2M) having molecular weight of 15 to 17 kDa and isoelectric point (PI) of 5.27 of 6.27, which comprises peptide sequences of SEQ ID NOs: 3 and 4 obtained by trypsin digestion thereof;

envelope protein having molecular weight of 27 to 29 kDa and isoelectric point (PI) of 6.5 to 7.5, which comprises peptide sequences of SEQ ID NOs: 5 and 6 obtained by trypsin digestion thereof;

SAP1 protein having molecular weight of 19 to 21 kDa and isoelectric point (PI) of 4.24 to 5.24, which comprises peptide sequence of SEQ ID NO: 7 obtained by trypsin digestion thereof;

lipocalin 1-like 1 having molecular weight of 20 to 23 kDa and isoelectric point (PI) of 4.13 to 5.43, which comprises peptide sequence of SEQ ID NO: 8 obtained by trypsin digestion thereof;

lipocalin (LCN-1; hCG201503) having molecular weight of 20 to 22 kDa and isoelectric point (PI) of 4.3 to 5.3, which comprises peptide sequence of SEQ ID NO: 8 obtained by trypsin digestion thereof;

cytokeratin having molecular weight of 20 to 22 kDa and isoelectric point (PI) of 4.64 to 5.64, which comprises peptide sequences of SEQ ID NOs: 9 to 14 obtained by trypsin digestion thereof;

S100 calcium-binding protein A8 having molecular weight of 21 to 23 kDa and isoelectric point (PI) of 6.01 to 7.01, which comprises peptide sequence of SEQ ID NO: 15 obtained by trypsin digestion thereof;

lipocalin 1 precursor having molecular weight of 21 to 23 kDa and isoelectric point (PI) of 4.89 to 5.89, which comprises peptide sequence of SEQ ID NO: 8 obtained by trypsin digestion thereof;

keratin 31 having molecular weight of 27 to 29 kDa and isoelectric point (PI) of 4.34 to 5.34, which comprises peptide sequences of SEQ ID NOs: 16 and 17 obtained by trypsin digestion thereof;

heat shock protein 27 (Hsp27) having molecular weight of 30 to 32 kDa and isoelectric point (PI) of 7.53 to 8.53, which comprises peptide sequences of SEQ ID NOs: 18 to 20 obtained by trypsin digestion thereof;

adenine phosphoribosyltransferase isoform having molecular weight of 19 to 21 kDa and isoelectric point (PI) of 5.28 to 6.28, which comprises peptide sequence of SEQ ID NO: 21 obtained by trypsin digestion thereof;

phosphohistidine phosphatase having molecular weight of 19 to 22 kDa and isoelectric point (PI) of 4.70 to 5.80, which comprises peptide sequence of SEQ ID NO: 22 obtained by trypsin digestion thereof;

beta globin having molecular weight of 18 to 21 kDa and isoelectric point (PI) of 5.96 to 6.96, which comprises peptide sequences of SEQ ID NOs: 23 to 25 obtained by trypsin digestion thereof;

lysozyme precursor having molecular weight of 18 to 20 kDa and isoelectric point (PI) of 6.5 to 7.5, which comprises peptide sequences of SEQ ID NOs: 26 and 27 obtained by trypsin digestion thereof;

S100 calcium-binding protein A9 having molecular weight of 14 to 16 kDa and isoelectric point (PI) of 5.21 to 6.21, which comprises peptide sequences of SEQ ID NOs: 28 to 30 obtained by trypsin digestion thereof;

crystalline human calprotectin having molecular weight of 13 to 15 kDa and isoelectric point (PI) of 5.21 to 6.21, which comprises peptide sequence of SEQ ID NO: 31 obtained by trypsin digestion thereof; and

S100 calcium-binding protein A4 having molecular weight of 11 to 13 kDa and isoelectric point (PI) of 6.28 to 7.28, which comprises peptide sequence of SEQ ID NO: 32 obtained by trypsin digestion thereof.

As used herein, the expression “peptide sequence obtained by trypsin digestion” means any peptide formed when a protein is digested by trypsin. Further, the molecular weight and isoelectric point (PI) of the proteins are measured by 2-dimensional electrophoresis, which may comprise an allowable experimental error.

As used herein, the term “non-retinopathic diabetic patient” means a patient suffering from diabetes mellitus (DM), e.g., type 2 DM without retinopathic symptoms, and the term “NPDR patient” means a patient suffering from type 2 DM with the symptoms of non-proliferative diabetic retinopathy (NPDR).

As used herein, the term “immunogenic fragment” means a fragment of a protein, which has at least one epitope being recognized by an antibody specific for the protein.

In one embodiment of the present invention, there is provided a use of a protein selected from the group consisting of DJ-1 protein and beta 2-microglobulin, or an immunogenic fragment thereof for diagnosing diabetic retinopathy, the expression level of the protein in tears of NPDR patients being higher than that of non-retinopathic diabetic patients. The expression level of these proteins or the immunogenic fragments thereof in the tears of NPDR patients may be at least 100%, preferably 200%, more preferably 300%, higher than that of non-retinopathic diabetic patients.

In another embodiment of the present invention, there is provided a use of a protein selected from the group consisting of the following proteins or an immunogenic fragment thereof for diagnosing diabetic retinopathy, the expression level of the protein in tears of NPDR patients being lower than that of non-retinopathic diabetic patients: envelope protein, SAP1, lipocalin 1-like 1, lipocalin (LCN-1; hCG201503), cytokeratin, S100 calcium-binding protein A8, lipocalin 1 precursor, keratin 31, heat shock protein 27, adenine phosphoribosyltransferase isoform, phosphohistidine phosphatase, beta globin, lysozyme precursor, S100 calcium-binding protein A9, crystalline human calprotectin, and S100 calcium-binding protein A4. The expression level of these proteins or the immunogenic fragments thereof in the tears of NPDR patients may be at least 30%, preferably 50%, lower than that of non-retinopathic diabetic patients.

In accordance with another aspect of the present invention, there is provided a composition for diagnosing diabetic retinopathy, comprising an antibody specific for a protein selected from the group consisting of the above-mentioned biomarker proteins or an immunogenic fragment thereof as an active ingredient.

In the present invention, the antibody may be a monoclonal antibody or a polyclonal antibody, preferably a monoclonal antibody.

In the present invention, the polyclonal antibody may be prepared by a conventional method well-known to those skilled in the art, e.g., by administering the protein or the immunogenic fragment thereof as an immunogen into a host.

The host may be a mammal, e.g., a mouse, a rat, a sheep and a rabbit. The immunogen may be administered by intramuscular, intraperitoneal or subcutaneous injection generally in combination with an adjuvant for improving antigenicity thereof. Then, blood samples are taken periodically from the host, and when the titer of desired antibody specific for the immunogen becomes high, blood is collected from the host and an antiserum is prepared therefrom. Polyclonal antibodies specific for the protein may then be purified from such antiserum.

In the present invention, the monoclonal antibody may be prepared by hybridoma technique well-known to those skilled in the art (Koeher and Milstein, (1975) Nature, 256:495).

In accordance with a further aspect of the present invention, there is provided a kit for diagnosing diabetic retinopathy, comprising an antibody specific for a protein selected from the group consisting of the above mentioned biomarker proteins or an immunogenic fragment thereof as an active ingredient.

In the present invention, the kit may be prepared by conventional methods well-known to those skilled in the art and may include the antibodies, buffers, stabilizing agents, inert proteins and the like. The antibody may be conjugated with a detectable marker such as radionuclides, fluorescors or enzymes.

Preferably, the kit comprises 1) the antibody specific for the biomarker protein or the immunogenic fragment thereof; 2) a secondary antibody conjugated with a marker which will develop a color upon reaction with a substrate thereof; 3) a solution comprising the substrate; 4) washing solutions for use in the respective steps; and 5) a solution to stop the color reaction.

The inventive kit is provided for diagnosing diabetic retinopathy by a qualitative or a quantitative analysis of the biomarker protein using antigen-antibody binding reaction. The antigen-antibody binding reaction may be analyzed using one of the known methods in the art, for instance, enzyme linked immunosorbent assay (ELISA), radio-immunoassay (RIA), sandwich ELISA, western blot on polyacrylamide gel, immuno-dot blotting assay, immuno-fluorescence assay (IFA), immuno-chemiluminescence assay, immuno-histochemical staining and immuno-chromatography (Rapid), wherein ELISA is preferred. In the present invention, the kit may be so provided that an ELISA is performed using a 96-well microtiter plate having a surface pre-coated with the antibody.

The marker conjugated to the secondary antibody may be any conventional colorant, preferably, HRP (horseradish peroxidase), alkaline phosphatase, coloid gold, FITC (poly L-lysine-fluoresceinisothiocyanate), RITC (rhodamine-B-isothiocyanate), or dye. In a preferred embodiment, an anti-rabbit IgG-HRP conjugate is used.

The substrate for a color reaction may be used in consideration of the marker, and exemplary substrates include TMB (3,3′,5,5′-tetramethyl bezidine), ABTS[2,2′-azino-bis(3-ethylbenzothiazoline-6-sulfonic acid)] or OPD (o-phenylenediamine). The substrate is preferably provided as its solution in a buffer (0.1 M NaAc, pH 5.5). The substrates such as TMB is decomposed by HRP, which is used as a marker of the secondary antibody conjugate, to form a colored deposit, and the concentration of the biomarker protein may be measured by determining the amount of the colored deposit with naked eyes.

The washing solution may comprise a phosphate buffer, NaCl and tween^{R} 20, a buffer containing 0.02M phosphate buffer, 0.13M NaCl and 0.05% tween^{R} 20 is preferred. The washing solution is used for washing an antigen-antibody complex formed after the reaction of the secondary antibody with an antigen-antibody complex which is formed between the biomarker protein in a sample and the antibody specific for the biomarker protein. The inventive kit may further comprise a blocking solution, which is preferably a phosphate buffer containing 0.1% bovine albumin serum (BSA). The solution to stop the color reaction (stop solution) is preferably 2N sulfate solution.

In accordance with a still further aspect, there is provided a composition for treating or preventing diabetic retinopathy, comprising as an active ingredient a protein selected from the group consisting of the above-mentioned biomarker proteins or an immunogenic fragment thereof, whose expression level in the tears of NPDR patients is lower than that of non-retinopathic diabetic patients: envelope protein, SAP1, lipocalin 1-like 1, lipocalin (LCN-1; hCG201503), cytokeratin, S100 calcium-binding protein A8, lipocalin 1 precursor, keratin 31, heat shock protein 27 (Hsp27), adenine phosphoribosyltransferase isoform, phosphohistidine phosphatase, beta globin, lysozyme precursor, S100 calcium-binding protein A9, crystalline human calprotectin, and S100 calcium-binding protein A4.

The inventive composition for treating or preventing diabetic retinopathy may be directly administered to the eyes of the NPDR patients in the form of ophthalmic liquid formulations such as solution, suspensions and ointments. The inventive composition may be prepared by a conventional method, by mixing the biomarker protein, or the immunogenic fragment thereof with pharmaceutically acceptable carriers, excipients or dilutents. The inventive composition may be formulated into an ophthalmic liquid formulation, which is administered dropwise to the eyes of the NPDR patients

A suitable dose of the composition for eye drop administration may be determined in light of various relevant factors including the condition to be treated, the age, sex and weight of the patient, and the severity of the patient's symptoms and, in case of an adult, it may be ranging from 0.1 to 0.5 mg per day.

The present invention also provides a method of diagnosing diabetic retinopathy, which comprises measuring the expression level of a protein selected from the group consisting of the following proteins or an immunogenic fragment thereof in the tears of a subject, and comparing the measured expression level to that measured in a healthy subject not suffering from diabetes: DJ-1 protein, beta 2-microglobulin, envelope protein, SAP1, lipocalin 1-like 1, lipocalin (LCN-1; hCG201503), cytokeratin, S100 calcium-binding protein A8, lipocalin 1 precursor, keratin 31, heat shock protein 27, adenine phosphoribosyltransferase isoform, phosphohistidine phosphatase, beta globin, lysozyme precursor, S100 calcium-binding protein A9, crystalline human calprotectin, and S100 calcium-binding protein A4.

Specifically, the method for diagnosing diabetic retinopathy of the present invention comprises 1) subjecting the tears obtained from a subject to a reaction with an antibody specific for the biomarker protein or the immunogenic fragment thereof, to obtain an antigen-antibody complex; 2) adding a secondary antibody conjugated with a marker, which will develop a color upon reaction with a substrate thereof and a solution comprising the substrate to detect the antigen-antibody complex by color reaction; and 3) comparing the detected amount of the protein or the immunogenic fragment thereof in the subject with that detected in a healthy subject not suffering from diabetes.

In step 1), the reaction of the tears of the subject with the antibody may be performed in a nitrocellulose membrane, or 96-well plate made of polyvinyl resin or polystyrene resin, or on a slide glass.

The subject may be diagnosed as having diabetic retinopathy when the expression level of a protein selected from the group consisting of DJ-1 protein and beta 2-microglobulin, or an immunogenic fragment thereof, in the subject's tears is higher by at least 100%, preferably at least 200%, and more preferably at least 300%, than that of non-retinopathic diabetic patients; or the expression level of a protein selected from the group consisting of envelope protein, SAP1, lipocalin 1-like 1, lipocalin (LCN-1; hCG201503), cytokeratin, S100 calcium-binding protein A8, lipocalin 1 precursor, keratin 31, heat shock protein 27, adenine phosphoribosyltransferase isoform, phosphohistidine phosphatase, beta globin, lysozyme precursor, S100 calcium-binding protein A9, crystalline human calprotectin, and S100 calcium-binding protein A4, or an immunogenic fragment thereof, in the subject's tears is lower by at least 30%, preferably at least 50%, than that of non-retinopathic diabetic patients.

Further, the present invention provides a method of treating or preventing diabetic retinopathy in a subject in need thereof, which comprises the step of administering a therapeutically effective amount of a protein selected from the group consisting of the following proteins, or an immunogenic fragment thereof, to the eyes of the subject: envelope protein, SAP1, lipocalin 1-like 1, lipocalin (LCN-1; hCG201503), cytokeratin, S100 calcium-binding protein A8, lipocalin 1 precursor, keratin 31, heat shock protein 27, adenine phosphoribosyltransferase isoform, phosphohistidine phosphatase, beta globin, lysozyme precursor, S100 calcium-binding protein A9, crystalline human calprotectin, and S100 calcium-binding protein A4.

The following Examples are intended to further illustrate the present invention without limiting its scope.

### Example 1

### Analysis of Tear Proteins by 2-Dimensional Electrophoresis

### Step 1: Sample Preparation

Tear fluid samples were obtained from the meniscus of 14 healthy persons (control), 10 patients suffering from type 2 DM without retinopathy (hereinafter, referred to as “non-retinopathic diabetic patients”), and patients suffering from type 2 DM with NPDR (hereinafter, referred to as “NPDR patients”) by using polyester wicks. The polyester wicks thus obtained were placed on the top of 1.5 ml of micropipet tips and centrifuged at 10,000 ppm for 5 min (Union-55R centrifuge, Hanil science, Korea). The concentration of the tear proteins was measured by the Bradford method, and then the tear proteins were stored at −70° C. until further use.

### Step 2: 2-Dimensional Electrophoresis (2-DE)

### (2-1) Isoelectrofocusing (IEF)

60 μg each of the tear proteins obtained in step 1 was subjected to an isoelectrofocusing. 450 μl of rehydration buffer solution (8M urea, 2% CHAPS, 13 mM DTT, 1% IPG buffer and Bromophenol blue (BPB)) was added to the tear protein sample. The mixture was loaded in a 24 cm strip holder, and Drystrips (Amerahsm Pharmacia Biotech, Uppsala, Sweden) having a pH ranging from 4 to 7 was placed in the strip holder. The strips were rehydrated for 5 hours without a current, and then, for another 5 hours with a current of 80V. Isoelectrofocusing (IEF) was carried out for a total of 100,000 Vhr using an IPGphore IEF system (Amerahsm Pharmacia Biotech, Uppsala, Sweden).

### (2-2) Sodium Dodecyl Sulfate Polyacrylamide Gel Electrophoresis (SDS-PAGE)

Following IEF separation, the gel strips obtained from (2-1) were equilibrated with 10 ml of the first equilibration solution (6M urea, 50 mM Tris-Cl buffer (pH 8.8), 20% glycerol, 2% SDS and 1% DTT) for 15 min, and the first equilibration solution was removed. The gel strips were then equilibrated again with 10 ml of the second equilibration solution (6M urea, 50 mM Tris-Cl buffer (pH 8.8), 30% glycerol, 2% SDS and 1.5% IAA) for 10 min. The strips were applied onto 11-16% gradient of polyacryl amide gel (25×30 cm), and subjected to a sealing with 0.5% agarose gel. SDS-PAGE was conducted by adding a buffer solution (24 mM Tris, 192 mM glycine and 0.1% SDS) to an Ettan DALT system (Amerahsm Pharmacia Biotech, Uppsala, Sweden) at a current of 70 V for 1 hour, 140 V for 2 hours, and 320 V for 5 hours.

### (2-3) Staining

In order to visualize the gels obtained in (2-2), a silver staining was conducted. The gels obtained in (2-2) were fixed in a mixture of 50% methanol, 12% acetic acid and 0.05% formaldehyde for at least 2 hours. The fixed gels were rinsed with 50% ethanol three times for 20 min each, then sensitized with 0.2% sodium thiosulfate for 1 min and followed by washing with D.W. The gels were immersed in a mixture of 0.1% silver nitrate and 0.075% formaldehyde (37%) for 20 min, and washed with D.W. The resulting gels were developed with a mixture of 6% sodium carbonate and 0.075% formaldehyde (37%) for 7 min, and 50% methanol and 12% acetic acid were added thereto to fix the resulting gels.

### (2-4) Image Analysis

In order to analyze images of the silver-stained gels, the gels were scanned by an Image Scanner (Amersham Pharmacia Bio-tech, Uppsala, Sweden) The patterns of the spots were automatically analyzed using Image-Master 2D Platinum version 6.0 (GE Healthcare, UK).

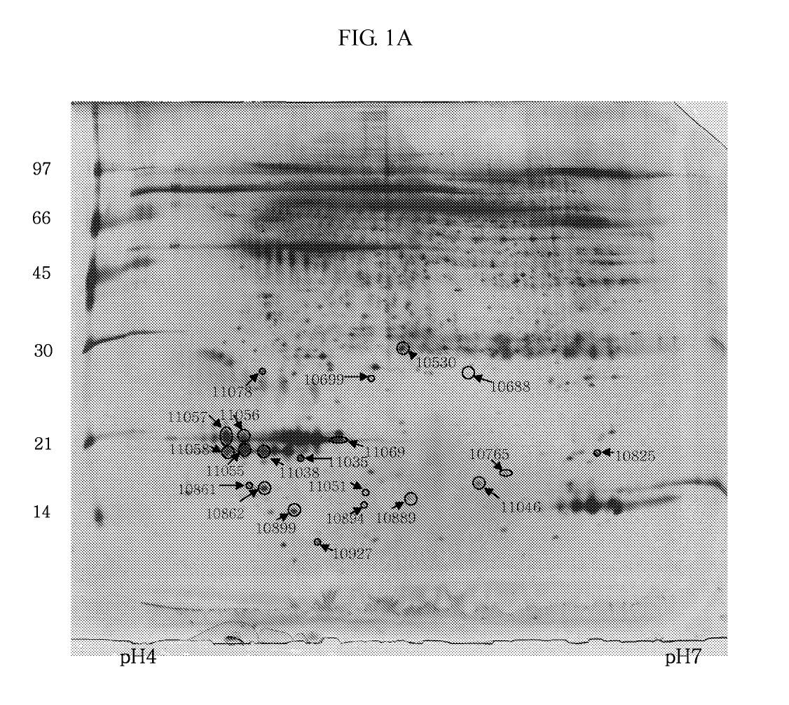

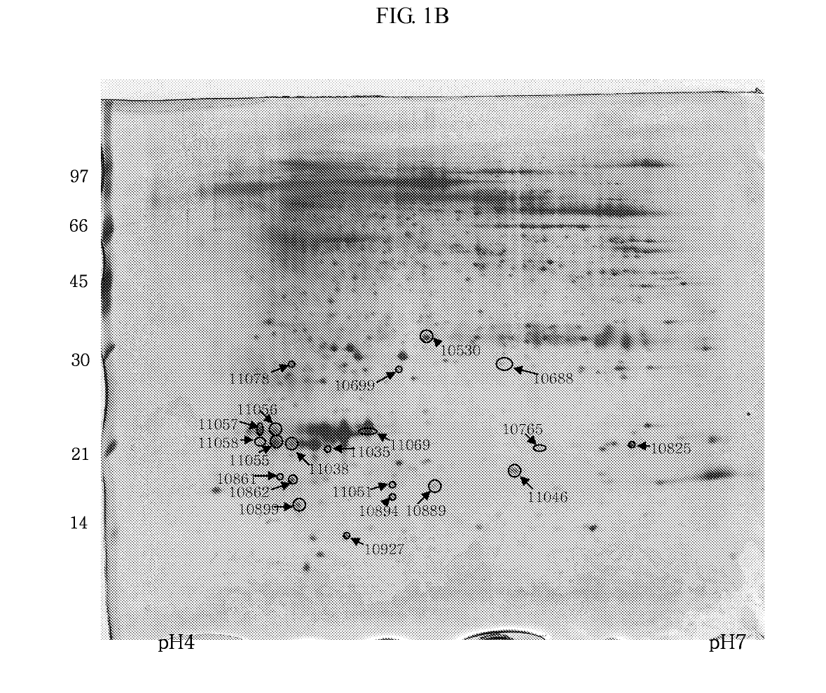

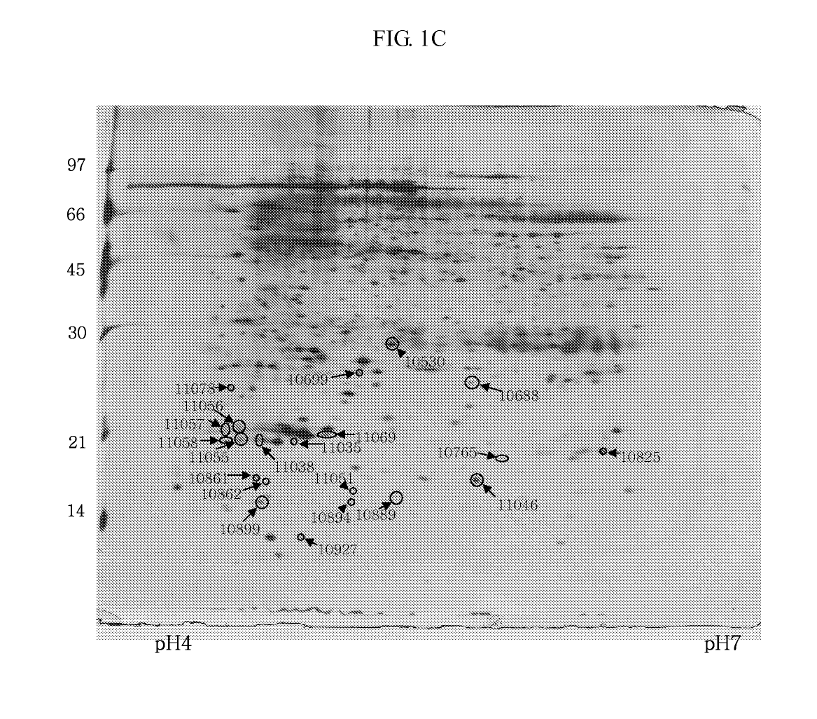

The images of SDS-PAGE electrophoresis of the proteins expressed in the tears of a healthy person (control), and a non-retinopathic diabetic patient and a NPDR patient are shown in FIGS. 1A to 1C, respectively.

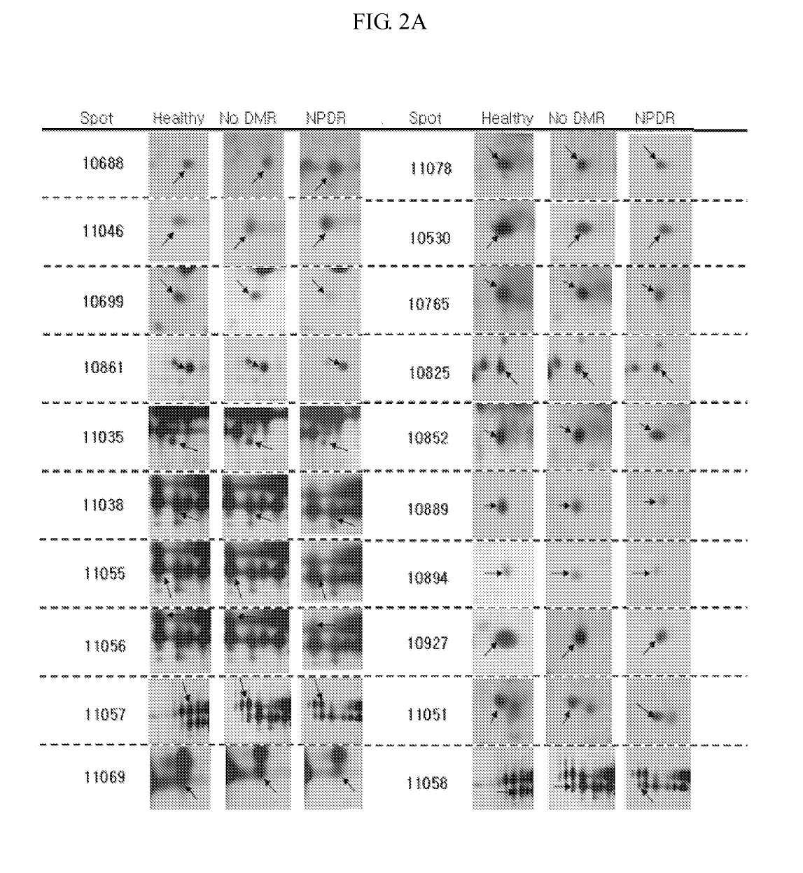

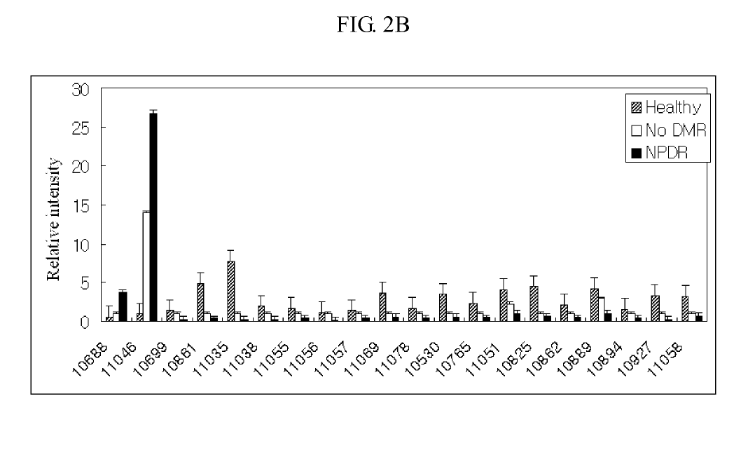

Also, the magnified images of the proteins shown in FIGS. 1A to 1C are shown in FIG. 2A, the relative intensities of the proteins are shown in FIG. 2B. Wherein Healthy is a healthy person group; No DMR is a non-retinopathic diabetic patient group; and NPDR is a NPDR patient group.

As shown in FIGS. 1A to 2B, newly expressed proteins and up-regulated or down-regulated proteins were observed in the samples of the NPDR patients as compared with those of the healthy persons or the non-retinopathic diabetic patients. Specifically, 18 proteins showing down-regulated expressions (spot Nos. of 10699, 10861, 11035, 11038, 11055, 11056, 11057, 11069, 11078, 10530, 10765, 11051, 10825, 10852, 10889, 10894, 10927 and 11058), and 2 proteins showing up-regulated expressions (spot Nos. 10688 and 11046) were found in the NPDR patients as compared with the healthy persons or non-retinopathic diabetic patients (see, FIGS. 1A to 2B). In the NPDR patients, the expression of 18 proteins were down-regulated by at least 50%, and that of 2 proteins were up-regulated by at least 300%, as compared with the healthy persons or the non-retinopathic diabetic patients.

The molecular weight and the isoelectric point (PI) of the spots of these proteins are shown in Table 1.

TABLE 1

| | Molecular weight | | |
|---|---|---|---|
| Spot No. | (kDa) | pI | classification |
| 10688 | 20/25 | 6.33 | Up-regulated |
| 11046 | 13/16 | 5.77 | Up-regulated |
| 10699 | 54/28 | 7 | Down-regulated |
| 10861 | 14/20 | 4.74 | Down-regulated |
| 11035 | 18/21 | 4.93 | Down-regulated |
| 11038 | 19/21 | 4.8 | Down-regulated |
| 11055 | 45/21 | 5.14 | Down-regulated |
| 11056 | 21/22 | 4.63 | Down-regulated |
| 11057 | 10/22 | 6.51 | Down-regulated |
| 11069 | 19/22 | 5.39 | Down-regulated |
| 11078 | 48/28 | 4.84 | Down-regulated |
| 10530 | 22/31 | 7.83 | Down-regulated |
| 10765 | 19/20 | 5.78 | Down-regulated |
| 11051 | 14/20 | 5.3 | Down-regulated |
| 10825 | 16/19 | 6.46 | Down-regulated |
| 10852 | 16/19 | 7 | Down-regulated |
| 10889 | 13/15 | 5.71 | Down-regulated |
| 10894 | 13/14 | 5.71 | Down-regulated |
| 10927 | 12/12 | 6.78 | Down-regulated |
| 11058 | 14/21 | 5.2 | Down-regulated |

### Example 2

### Identification of Proteins by ESI-Q-TOF MS/MS Analysis

To identify the proteins obtained in Example 1, the gel spots were excised. The excised gel spots were destained with 100 μL of destaining solution comprising 30 mM of potassium ferricyanide and 100 mM sodium thiosulfate as a ratio of 1:1 (v/v) for 5 min. After the gel spots were washed three times with 400 μl of water, the gel spots were incubated with 200 mM ammonium bicarbonate for 10 min, and washed again. The resulting gel spots were dehydrated with acetonitrile, until the gel was changed to be a white, and dried with a speed vacuum centrifuge. The dried gel spots were the rehydrated with 20 μl of 50 mM ammonium bicarbonate containing 0.2 μg of trypsin (promega) for 45 min on ice. After the removal of the solution, 30 μl of 50 mM ammonium bicarbonate was added thereto, and incubated overnight at 37° C. for digestion. A peptide solution obtained in above was desalted using C¹⁸ nano column (homemade).

Custom-made chromatographic columns were employed for desalting and concentration of the peptide solution, prior to mass spectrometric analysis. A column consisting of 100-300 mL of Poros reverse phase R2 material (20-30 um bead size, PerSeptive Biosystems) was packed into a constricted GELoader tip (Eppendorf, Hamburg, Germany). A 10 mL syringe was then utilized to force liquid through the column, by applying a gentle air pressure. 30 μl of the peptide solution from the digestion supernatant was then diluted, loaded onto the column, and washed with 20 μl of 5% formic acid. For MS/MS analysis, the peptides were eluted with a mixture of 50% methanol, 49% H₂O and 1% formic acid directly into a precoated borosilicate nanoelectrospray needle (Micromass, Manchester, U.K.).

The MS/MS analysis of the peptides was performed by nano-ESI on a Q-TOF mass spectrometer (Micromass, Manchester, U.K.). The source temperature was 80° C. A 1 kV potential was applied to precoated borosilicate nanoelectrospray needles (EconoTip, New Objective, USA) in the ion source, combined with a nitrogen back-pressure of 0-5 psi, to achieve a stable flow rate (10-30 nL/min). The cone voltage was 40 V. The quadrupole analyzer was used to select precursor ions for fragmentation in the hexapole collision cell. The collision gas used was argon (Ar) at a pressure of 6-7×10⁻⁵ mbar, and a collision energy was 20-30V. The product ions were analyzed with an orthogonal TOF analyzer, fitted with a reflector, a microchannel plate detector and a time-to-digital converter. The data was processed using a Mass Lynx Windows NT PC system (Micromass, Manchester, U.K.).

For identification of the protein, all MS/MS spectra recorded on the tryptic peptides derived from the spots were searched against the protein sequences from the NCBInr databases (www.ncbi.nlm.nih.gov), using the MASCOT search program (www.matrixscience.com). The results of identification of the proteins up-regulated or down-regulated, are shown in Tables 2 and 3, respectively.

TABLE 2

| | | | Peptide by trypsin digestion | | | |
|---|---|---|---|---|---|---|
| | | | | SEQ | NCBI | SEQ |
| Spot | Identification | MW | | ID | Assession | ID |
| NO. | of protein | (Da) | Sequence | NO. | No. | NO. |
| 10688 | DJ-1 protein | 20050 | GAEEMETVIPVDVMR | 1 | NP_009193 | 33 |
| | | | EGPYDVVVLPGGNLGAQNLSESAAVK | 2 | | |
| 11046 | Beta-2 | 12905 | SNFLNCYVSGFHPSDIEVDLLK | 3 | CAA23830 | 34 |
| | microglobulin | | DWSFYLLYYTEFTPTEKDEYACR | 4 | | |

TABLE 3

| | | | Peptide by trypsin digestion | | | |
|---|---|---|---|---|---|---|
| | | | | SEQ | NCBI | SEQ |
| Spot | Identification | MW | | ID | Assession | ID |
| NO. | of protein | (Da) | Sequence | NO. | No. | NO. |
| 10699 | Envelope protein | 54093 | MINIEASQLAEVR | 5 | AAB05390 | 35 |
| | | | SGLNTEAFYVMTVGSK | 6 | | |
| 10861 | Protein SAP1 | 13491 | IIPGGIYDADLNDEWVQR | 7 | AAB19889 | 36 |
| 11035 | Lipocalin 1-like 1 | 18078 | GLSTESILIPR | 8 | CAH73781 | 37 |
| 11056 | Lipocalin 1-like 1 | 18078 | GLSTESILIPR | 8 | CAH73781 | 37 |
| 11038 | Lipocalin | 19488 | GLSTESILIPR | 8 | EAW88053 | 38 |
| | (hCG201503) | | | | | |
| 11055 | Cytokeratin 4 | 45593 | FASFIDKVQFLEQQNK | 9 | CAA30534 | 39 |
| | | | WNLLQQQTTTTSSK | 10 | | |
| | | | NLEPLFETYLSVLR | 11 | | |
| | | | VDSLNDEINFLK | 12 | | |
| | | | NLDLDSIIAEVR | 13 | | |
| | | | VQQLQISVDQHGDNLK | 14 | | |
| 11057 | S100 calcium- | 10885 | ELDINTDGAVNFQEFLILVIK | 15 | NP_002955 | 40 |
| | binding protein | | | | | |
| | A8 | | | | | |
| 11069 | Lipocalin 1 | 19409 | GLSTESILIPR | 8 | NP_002288 | 41 |
| | precursor | | | | | |
| 11078 | Keratin 4 | 48633 | QNQEYQVLLDVR | 16 | NP_002268 | 42 |
| | | | LNVEVDAAPTVDLNR | 17 | | |
| 10530 | Heat shock | 22427 | LFDQAFGLPR | 18 | AAA62175 | 43 |
| | protein 27 | | VSLDVNHFAPDELTVK | 19 | | |
| | | | LATQSNEITIPVTFESR | 20 | | |
| 10765 | Adenine | 19766 | LQAEVLECVSLVELTSLK | 21 | NP_000476 | 44 |
| | phospho-ribosyl- | | | | | |
| | transferase | | | | | |
| | isoform | | | | | |
| 11051 | Phosphohistidine | 14 kDa | ALIPDVDLDSD | 22 | BC024648 | 45 |
| | phosphatase | | | | | |
| 11058 | Phosphohistidine | 14 kDa | ALIPDVDLDSD | 22 | BC024648 | 45 |
| | phosphatase | | | | | |
| 10825 | Beta globin | 16101 | LLVVYPWTQR | 23 | AAF00488 | 46 |
| | | | EFTPPVQAAYQK | 24 | | |
| | | | FFESFGDLSTPDAVMGNPK | 25 | | |
| 10852 | Lysozyme | 16885 | GISLANWMCLAK | 26 | 1LHM | 47 |
| | precursor | | STDYGIFQINSR | 27 | | |
| 10889 | S100 calcium | 13291 | QLSFEEFIMLMAR | 28 | NP_002956 | 48 |
| | binding protein | | NIETIINTFHQYSVK | 29 | | |
| | A9 | | VIEHIMEDLDTNADKQLSFEEFIMLMAR | 30 | | |
| 10894 | Crystal structure | 13086 | QLSFEEFIMLMAR | 31 | NP_002956 | 49 |
| | of human | | | | | |
| | calprotectin | | | | | |
| 10927 | S100 calcium | 11949 | ALDVMVSTFHK | 32 | AAB20971 | 50 |
| | binding protein | | | | | |
| | A4 | | | | | |

### Example 3

### Western Blotting

### (3-1) SDS-PAGE Electrophoresis

40-50 μg of the proteins obtained from the tears of the non-retinopathic diabetic patients and NPDR patients were diluted with SDS-PAGE loading buffer (60 mM Tris-Cl, 2% SDS, 25% Glycerol, 14.4 mM 2-mercaptoethanol and 0.1% bromophenol blue; pH 6.8), and subjected to 12% SDS-PAGE. The gels were blotted onto mini gel (6×8 cm) using a running buffer at a current of 120V for molecular weight separation. The running buffer is containing 0.025M Tris-Cl, 0.192M Glycine and 1% SDS (pH 8.3).

### (3-2) Blotting

The gels obtained in (3-1) were transferred onto nitrocellulose (NC) membranes to blot using semi-dry transfer kit (Bio-rad) at a current of 50 mA for 1 hour 30 min.

### (3-3) Antibody Reaction

An antibody reaction was conducted by employing LCN-1 (Spot NO. 11038), Hsp27 (Spot NO. 10530) and B2M (Spot NO. 11046) obtained from the NPDR patients. The nitrocellulose membranes were incubated with blocking solution of 5% w/v nonfat dry milk for 1 hour at room temperature. Anti-LCN1-monoclonal (1:1000), Anti-Hsp27-monoclonal (1:1000) and Anti-B2M-monoclonal (1:500) antibodies were used with the blocking solution, and incubated at 4° C. for 24 hours. The monoclonal antibody prepared by a conventional hybridoma technique (Koeher and Milsteinas (1975) Nature, 256:495) using a rabbit was used as a primary antibody. The resulting solution was washed with TBS/T (0.1% Tris-buffered saline-tween20), and subsequently incubated with a solution containing 1:10,000 of horseradish peroxidase (HRP)-conjugated anti-rabbit secondary antibody (Santa cruz biotechnology Incs.) at a room temperature for 1 hour. The resulting solution was washed with TBS/T, and detection was performed with Enhanced chemiluminescence (ECL) system (Pierce Biotechnology Inc., USA).

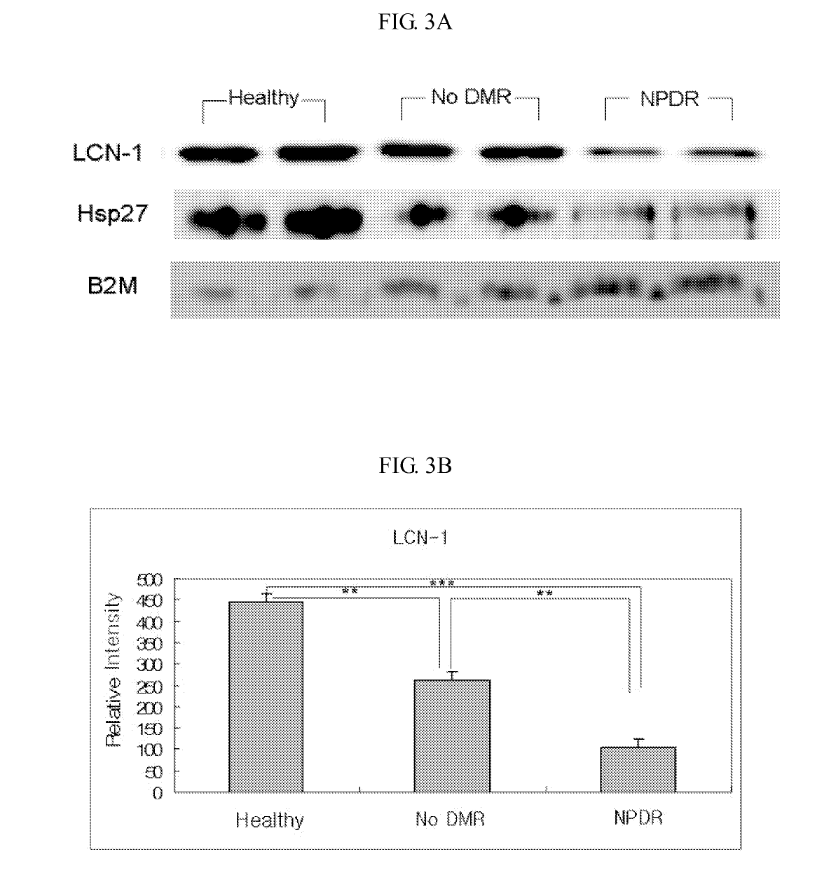

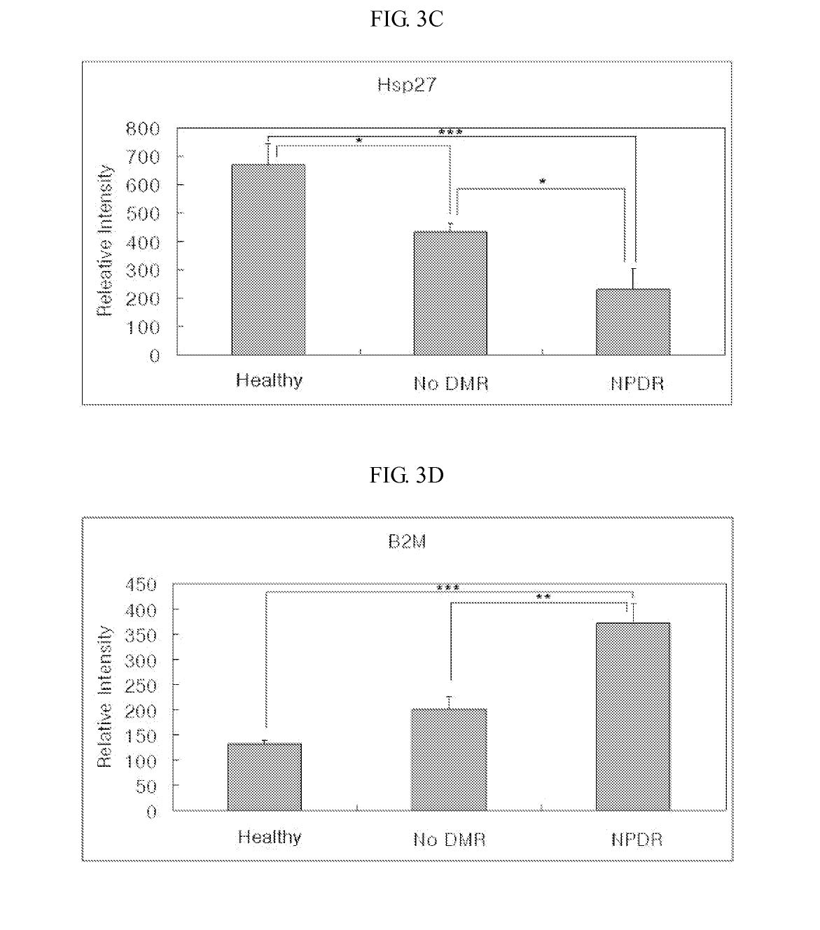

The results of western blot analysis of LCN-1, Hsp27 and B2M are shown in FIG. 3A, and relative intensities of the proteins are shown in FIGS. 3B to 3D, respectively (In each bar, *, ** and *** represents that significant differences between groups determined by unpaired student's t-test are p<0.05, p<0.01 and p<0.001, respectively).

As shown in FIGS. 3A to 3D, the expression levels of LCN-1 and Hsp 27 were significantly down-regulated, and the expression level of B2M was up-regulated according to the progress of diabetic retinopathy. These results corresponds to the results of 2-DE. Therefore, it is concluded that LCN-1, Hsp27 and B2M are useful as biomarkers for diagnosing diabetic retinopathy, and are useful treating or preventing diabetic retinopathy.

While the invention has been described with respect to the above specific embodiments, it should be recognized that various modifications and changes may be made to the invention by those skilled in the art which also fall within the scope of the invention as defined by the appended claims.

What is claimed is:

1. A method of diagnosing diabetic retinopathy, which comprises:
(a) measuring an expression level of beta 2-microglobulin (B2M) or an immunogenic fragment thereof in the tears of a subject, said B2M having molecular weight of 15 to 17 kDa and isoelectric point (PI) of 5.27 to 6.27 and comprising peptide sequences of SEQ ID NOs: 3 and 4 obtained by trypsin digestion thereof; and
(b) comparing the measured expression level to that measured in a healthy subject not suffering from diabetes.

2. The method of claim 1, wherein measuring the expression level of B2M in step (a) comprises
i) subjecting the tears obtained from a subject to a reaction with an antibody specific for said B2M or the immunogenic fragment thereof to obtain an antigen-antibody complex; and
ii) adding to the antigen-antibody complex a secondary antibody conjugated with a marker, which develops a color upon reaction with a substrate thereof, and a solution comprising the substrate to detect the antigen-antibody complex by color reaction, thereby measuring the expression level.

3. The method of claim 2, wherein the antibody is a monoclonal antibody.


SEQUENCE LISTING (50 sequences)

SEQ ID NO: 1 - protein, 15 aa, Homo sapiens
GAEEMETVIPVDVMR

SEQ ID NO: 2 - protein, 26 aa, Homo sapiens
EGPYDVVVLPGGNLGAQNLSESAAVK

SEQ ID NO: 3 - protein, 22 aa, Homo sapiens
SNFLNCYVSGFHPSDIEVDLLK

SEQ ID NO: 4 - protein, 23 aa, Homo sapiens
DWSFYLLYYTEFTPTEKDEYACR

SEQ ID NO: 5 - protein, 13 aa, Homo sapiens
MINIEASQLAEVR

SEQ ID NO: 6 - protein, 16 aa, Homo sapiens
SGLNTEAFYVMTVGSK

SEQ ID NO: 7 - protein, 18 aa, Homo sapiens
IIPGGIYDADLNDEWVQR

SEQ ID NO: 8 - protein, 11 aa, Homo sapiens
GLSTESILIPR

SEQ ID NO: 9 - protein, 16 aa, Homo sapiens
FASFIDKVQFLEQQNK

SEQ ID NO: 10 - protein, 14 aa, Homo sapiens
WNLLQQQTTTTSSK

SEQ ID NO: 11 - protein, 14 aa, Homo sapiens
NLEPLFETYLSVLR

SEQ ID NO: 12 - protein, 12 aa, Homo sapiens
VDSLNDEINFLK

SEQ ID NO: 13 - protein, 12 aa, Homo sapiens
NLDLDSIIAEVR

SEQ ID NO: 14 - protein, 16 aa, Homo sapiens
VQQLQISVDQHGDNLK

SEQ ID NO: 15 - protein, 21 aa, Homo sapiens
ELDINTDGAVNFQEFLILVIK

SEQ ID NO: 16 - protein, 12 aa, Homo sapiens
QNQEYQVLLDVR

SEQ ID NO: 17 - protein, 15 aa, Homo sapiens
LNVEVDAAPTVDLNR

SEQ ID NO: 18 - protein, 10 aa, Homo sapiens
LFDQAFGLPR

SEQ ID NO: 19 - protein, 16 aa, Homo sapiens
VSLDVNHFAPDELTVK

SEQ ID NO: 20 - protein, 17 aa, Homo sapiens
LATQSNEITIPVTFESR

SEQ ID NO: 21 - protein, 18 aa, Homo sapiens
LQAEVLECVSLVELTSLK

SEQ ID NO: 22 - protein, 11 aa, Homo sapiens
ALIPDVDLDSD

SEQ ID NO: 23 - protein, 10 aa, Homo sapiens
LLVVYPWTQR

SEQ ID NO: 24 - protein, 12 aa, Homo sapiens
EFTPPVQAAYQK

SEQ ID NO: 25 - protein, 19 aa, Homo sapiens
FFESFGDLSTPDAVMGNPK

SEQ ID NO: 26 - protein, 12 aa, Homo sapiens
GISLANWMCLAK

SEQ ID NO: 27 - protein, 12 aa, Homo sapiens
STDYGIFQINSR

SEQ ID NO: 28 - protein, 13 aa, Homo sapiens
QLSFEEFIMLMAR

SEQ ID NO: 29 - protein, 15 aa, Homo sapiens
NIETIINTFHQYSVK

SEQ ID NO: 30 - protein, 28 aa, Homo sapiens
VIEHIMEDLDTNADKQLSFEEFIMLMAR

SEQ ID NO: 31 - protein, 13 aa, Homo sapiens
QLSFEEFIMLMAR

SEQ ID NO: 32 - protein, 11 aa, Homo sapiens
ALDVMVSTFHK

SEQ ID NO: 33 - protein, 188 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDLILTSRGPGTSFEFALAIVEALNGKEVAAQVKAPLVLKD

SEQ ID NO: 34 - protein, 110 aa, Homo sapiens
LALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 35 - protein, 500 aa, Homo sapiens
FNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGLFGKGSIDTCAKFSCTSKAIGRTIQPENIKYEVGIFVHGTTTSENHGNYSAQVGASQAAKFTVTPNAPSITLKLGDYGEVTLDCEPRSGLNTEAFYVMTVGSKSFLVHREWFHDLALPWTSPSSTAWRNRELLMEFEEAHATKQSVVALGSQEGGLHQALAGAIVVEYSSSVKLTSGHLKCRLKMDKLALKGTTYGMCTEKFSFAKNPADTGHGTVVIELSYSGSDGPCKIPIVSVASLNDMTPVGRLVTVNPFVATSSANSKVLVEMEPPFGDSYIVVGRGDKQINHHWHKAGSTLGKAFSTTLKGAQRLAALGDTAWDFGSIGGVFNSIGKAVHQVFGGAFRTLFGGMSWITQGLMGALLLWMGVNARDRSIALAFLATGGVLVFLATNVHA

SEQ ID NO: 36 - protein, 113 aa, Homo sapiens [sequence omitted]

SEQ ID NO: 37 - protein, 162 aa, Homo sapiens
MKPLLLAISLSLIAALQAHHLLASDEEIQDVSGTWYLKAMTVDRELPEMNLESVTPMTLTILEGGNLEAKATMLISGQCQEVKVILEKTDEPGKYTANRGKHVAYIIRSHMKDHYIFYCEGRDPENNLEALEDFEKAAGARGLSTESILIPRQSETCSPGSD

SEQ ID NO: 38 - protein, 176 aa, Homo sapiens [sequence omitted]

SEQ ID NO: 39 - protein, 408 aa, Homo sapiens
SLNQSLLTPLHVEIDPEIQKVRTEEREQIKLLNNKFASFIDKVQFLEQQNKVLETKWNLLQQQTTTTSSKNLEPLFETYLSVLRKQLDTLGNDKGRLQSELKTMQDSVEDFKTKYEEEINKRTAAENDFVVLKKDVDAAYLNKVELEAKVDSLNDEINFLKVLYDAELSQMQTHVSDTSVVLSMDNNRNLDLDSIIAEVRAQYEEIAQRSKAEAEALYQTKVQQLQISVDQHGDNLKNTKSEIAELNRMIQRLRAEIENIKKQCQTLQVSVADAEQRGENALKDAHSKRVELEAALQQAKEELARMLREYQELMSVKLALDIEIATYRKLLEGEEYRMSGECQSAVSISVVSGSTSTGGISGGLGSGSGFGLSSGFGSGSGSGFGFGGSVSGSSSSKIISTTTLNKRR

SEQ ID NO: 40 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 41 - protein, 176 aa, Homo sapiens
MKPLLLAVSLGLIAALQAHHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 42 - protein, 416 aa, Homo sapiens [sequence omitted]

SEQ ID NO: 43 - protein, 199 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGRSCKIR

SEQ ID NO: 44 - protein, 180 aa, Homo sapiens
MADSELQLVEQRIRSFPDFPTPGVVFRDISPVLKDPASFRAAIGLLARHLKATHGGRIDYIAGLDSRGFLFGPSLAQELGLGCVLIRKRGKLPGPTLWASYSLEYGKAELEIQKDALEPGQRVVVVDDLLATGGTMNAACELLGRLQAEVLECVSLVELTSLKGREKLAPVPFFSLLQYE

SEQ ID NO: 45 - protein, 125 aa, Homo sapiens
MAVADLALIPDVDIDSDGVFKYVLIRVHSAPRSGAPAAESKEIVRGYKWAEYHADIYDKVSGDMQKQGCDCECLGGGRISHQSQDKKIHVYGYSMAYGPAQHAISTEKIKAKYPDYEVTWANDGY

SEQ ID NO: 46 - protein, 147 aa, Homo sapiens [sequence omitted]

SEQ ID NO: 47 - protein, 148 aa, Homo sapiens [sequence omitted]

SEQ ID NO: 48 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 49 - protein, 114 aa, Homo sapiens; identical to SEQ ID NO: 48

SEQ ID NO: 50 - protein, 101 aa, Homo sapiens
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK